(19) United States
(12) Patent Application Publication
(10) Pub. No.: US 2023/0346651 A1
(43) Pub. Date: Nov. 2, 2023

(54) SURFACTANT-FREE FOAMABLE ORAL CARE COMPOSITION

(71) Applicant: SHANDONG BENZHEN COSMETICS CO., LTD., Shandong (CN)

(72) Inventors: Zhengmei HUANG, Shandong (CN); Jinping DING, Shandong (CN); Hui LIN, Shandong (CN)

(73) Assignee: SHANDONG BENZHEN COSMETICS CO., LTD., Shandong (CN)

(21) Appl. No.: 18/026,694

(22) PCT Filed: Feb. 3, 2021
(86) PCT No.: PCT/CN2021/075095

§ 371 Date: Mar. 16, 2023

(30) Foreign Application Priority Data
Sep. 23, 2020 (CN) 202011011010.7

(51) Int. Cl. A61K 8/04; A61Q 11/00; A61K 8/73; A61K 8/46; A61K 8/44; A61K 8/60

(52) U.S. Cl. CPC A61K 8/046; A61Q 11/00; A61K 8/731; A61K 8/463; A61K 8/44; A61K 8/602; A61K 2800/28; A61K 2800/30

(57) ABSTRACT

A surfactant-free foamable oral care composition is provided. The oral care composition includes 0.1-10% (w/w) of methyl cellulose, a surface tension of an aqueous solution of the 0.1% methyl cellulose is less than 60 mN/m. The oral care composition includes no surfactant, while can generate foams during tooth brushing.

### CROSS REFERENCE TO RELATED APPLICATION

This application claims the priority of the Chinese patent application filed in China Patent Office on Sep. 23, 2020, with the application number of CN2020110111010.7, the entire contents of which are incorporated herein by reference.

### FIELD OF TECHNOLOGY

The present disclosure relates to an oral care composition, particularly to a surfactant-free oral care composition, in particular to a surfactant-free foamable toothpaste composition.

### BACKGROUND

Toothpaste is a daily necessity for people. As a product in close contact with the oral cavity every day, the safety of toothpaste and its influence on human health are becoming the attention focus of people. Toothpaste is a mixture with complex components, and is generally prepared by mixing an abrasive, a humectant, a surfactant, a thickener, a flavoring essence, a sweetener, a coloring agent and a bactericidal preservative, etc.

The surfactant is a necessary component in a toothpaste product. For example, U.S. Pat. No. 4,058,595 discloses surfactants commonly used in toothpaste, such as alkylaryl sulfonate, higher alkyl sulphoacetate, higher fatty acid amides of taurine and sorbitan monostearate, etc. However, these synthetic surfactants and their decomposition products may have irritation to mucosa, skin allergy, toxicity and carcinogenicity, and may destroy the ecological environment of the skin and oral cavity, including destroying skin taste cells and oral cavity cells, and the residual chemical components may destroy the ecological environment of the oral cavity, even permeate through the oral mucosa to enter blood, thus bringing serious potential safety hazards. Therefore, the safety of the toothpaste is widely concerned by consumers.

To reduce the potential safety hazards of the surfactants, a type of mild toothpaste has also been developed, this type of mild toothpaste uses relatively mild surfactants, such as sodium lauroyl sarcosinate, alkyl glucosides, cocamidopropyl betaine and natural surfactants (soyasaponin), etc. However, our research team performed mung bean sprouting experiments on a toothpaste-water solution with a certain concentration in a laboratory. Results show that mung beans could not sprout even after the surfactant-containing toothpaste was diluted by 20 times with water. Researches show that due to the existence of the surfactant, the safety of the toothpaste is reduced, and potential safety hazards exist.

Additionally, alternatives to surfactants have also been developed for use in toothpaste compositions.

CN103211716A discloses a method for manufacturing toothpaste by using a protein with surface activity or a protein-based surfactant as a unique cleaning and washing component, wherein the protein with surface activity or the protein-based surfactant is one, two, three or four of sodium soybean protein, calcium silk protein, a natural silk protein emulsifier or sodium caseinate.

US2006222602A1 discloses a toothpaste composition for cleaning teeth. A calcium salt component of the toothpaste composition includes nanoparticles and a protein component such as soybean protein and casein, etc. The toothpaste composition further includes an anion surfactant, a zwitter ionic and amphoteric surfactant, a nonionic surfactant, a cation surfactant or a mixture of these compounds. The toothpaste composition has effects of soft treatment and cleaning.

CN108261343A discloses a toothpaste composition including 0.05-5.5% of calcium caseinate based on the total weight of the toothpaste composition, but the cost of this alternative surfactant is high.

In order to reduce the potential safety hazards caused by the surfactant, a type of surfactant-free toothpaste is also developed, but the surfactant-free toothpaste cannot generate foams during tooth brushing, so that the experience feelings of users are poor, and the acceptance degree of the users is low.

Therefore, there is still a need to develop a surfactant-free and preservative-free oral care composition, particularly a toothpaste composition, which does not reduce the use experience feeling.

### SUMMARY

Cellulose type naturally sourced high-molecular polymers are a common thickener in toothpaste. Researchers of the present disclosure surprisingly found that an oral care composition using specific methyl cellulose as a thickener can still generate foams under a surfactant-free condition, and completed the present disclosure on this basis.

Therefore, the present disclosure relates to a foamable oral care composition, includes 0.1-10 weight % of a methyl cellulose by the weight of the oral care composition, a surface tension of the methyl cellulose in an aqueous solution at a concentration of 0.1 mass % is less than 60 mN/m, preferably is 47-59 mN/m, especially preferably 48-53 mN/m. It should be understood that the component content in the composition of present disclosure is mass percent (w/w).

In accordance with some embodiments, the present disclosure relates to an oral care composition, includes:
(a) 10-50% (w/w) of an abrasive, preferably 12-40%, especially preferably 15-30%, and particularly 18-25%;
(b) 5-50% (w/w) of a humectant, preferably 10-45%, especially preferably 15-40%, and particularly 18-30%;
(c) 0.01-2.0% (w/w) of a flavoring agent, preferably 0.1-1.0%, and more preferably 0.5-1.0%;
(d) 0.01-10% (w/w) of a functional additive, preferably 0.1-5%, and more preferably 1.0-2.0%;
(e) 0.1-10% (w/w) of methyl cellulose, preferably 0.5-10%, more preferably 1-7%, especially preferably 2-5%, and particularly 2.5-4.0%.

In at least one or more embodiments, the oral care composition of the present disclosure includes:
(a) 10-50% of an abrasive, preferably 12-40%, especially preferably 15-30%, and particularly 18-25%;
(b) 5-50% of a humectant, preferably 10-45%, especially preferably 15-40%, and particularly 18-30%;
(c) 2-20% of cellulose, preferably 4-16%, especially preferably 6-12%, and particularly 8-10%;
(d) 5-30% of a lactobacillus fermentation compound, preferably 8-26%, especially preferably 12-22%, and particularly 14-18%;
(e) 0.1-10% of methyl cellulose, preferably 0.5-10%, more preferably 1-7%, especially preferably 2-5%, and particularly 2.5-4.0%; and
(f) 1-12% of a starch, preferably 2-10%, especially preferably 3-8%, and particularly 4-6.0%. Methyl cellulose (MC)

In the oral care composition of the present disclosure, a surface tension of an aqueous solution of 0.1% methyl cellulose is less than 60 mN/m, preferably 47-59 mN/m.

In order to obtain an acceptable foam effect, based on the oral care composition, a consumption of the methyl cellulose of the present disclosure is 0.1-10% (w/w), preferably 0.5-10%, more preferably 1-7%, especially preferably 2-5%, and particularly 2.5-4.0%.

In some embodiments, commercially available methyl cellulose is selected, such as 55HD400FG(food grade) and 55HD400(medical grade) of Shandong HEAD Company.

Abrasive

In a plurality of embodiments of the present disclosure, under the condition that a medium of the oral care composition is solid or paste, the oral composition includes a dental acceptable abrasive material or polishing agent which can be used for polishing tooth enamel or providing a whitening effect. Any oral acceptable abrasive can be used. However, the type, the fineness (particle size) and the quantity of the abrasive should be selected so that the tooth enamel is not excessively abraded in normal use of the composition. A proper abrasive includes, but is no limited to silicon dioxide, such as silica gel, silicon dioxide hydrate or precipitated silicon dioxide forms, aluminum oxide, insoluble phosphate, calcium carbonate and a resin abrasive such as urea formaldehyde condensates, etc. The insoluble phosphate used as the abrasive includes orthophosphate, polymetaphosphate and pyrophosphate. Exemplary examples are calcium orthophosphate dihydrate, calcium pyrophosphate, (3-calcium pyrophosphate, tricalcium phosphate, calcium polymetaphosphate and insoluble sodium polymetaphosphate. One or more abrasives optionally exist at an effective total abrasion amount of typically about 10-50%, for example 12-40%, 15-30%, or 18-25% of the composition.

Humectant

The humectants capable of being used in the present disclosure include polyhydric alcohol, such as glycerol, sorbitol, xylitol and polyethylene glycol. In various embodiments, the humectant can be used for preventing paste or a gel composition from hardening when being exposed in the air, the humectant also achieves the effect of a sweetening agent. One or more humectants optionally exist at a total amount of 5-50%, preferably 10-45%, especially preferably 15-40%, and particularly 18-30%.

Thickener

The thickener is generally used for the oral care composition so as to control or change the viscosity of the composition. On the basis of not influencing the foams of the toothpaste, the toothpaste in at least one embodiment of the present disclosure additionally includes a thickener different from the methyl cellulose and selected from: polysaccharide or polysaccharide derivatives (such as hydroxypropyl methyl cellulose, ethyl cellulose, carboxymethyl cellulose, hydroxyethyl cellulose, hydroxypropyl cellulose and other cellulose derivatives and a starch), carbomers (such as crosslinked polyacrylic acid copolymers or acrylic acid homopolymers and copolymers crosslinked with polyalkenyl polyether), natural and synthetic gums (such as carrageenan, xanthan gum, karaya gum, guar gum, gelatin, algin, sodium alginate, tragacanth gum, chitosan and acacia gum), acrylamide polymers, acrylic acid polymers, vinyl polymers (such as polyvinyl alcohol and polyvinyl pyrrolidone), polyamine, polyquaternary compounds, ethylene oxide polymers, and a mixture thereof. In some embodiments, clay, organically modified clay, some inorganic thickeners such as silicon dioxide and a mixture thereof are used for the present disclosure.

Flavoring Agent

The flavoring agent used in the present disclosure includes any substance capable of being operated to enhance the taste of the composition, or a mixture of the substances. Any oral acceptable natural or synthetic flavoring agent can be used, such as flavoring oil, flavoring aldehyde, ester, alcohol, similar substances, and a combination thereof. The flavoring agent includes vanillin, sage, marjoram, parsley oil, spearmint oil, cinnamon oil, wintergreen oil, peppermint oil, clove oil, bay oil, anise oil, eucalyptus oil, citrus oil, fruit oil and essences derived from lemons, oranges, limes, grapefruits, apricots, bananas, grapes, apples, strawberries, cherries and pineapples, etc. One or more flavoring agents optionally exist at a total amount of 0.01-5%.

In some embodiments, the flavoring agent used in the present disclosure includes an oral acceptable natural or artificial nutritive or non-nutritive sweetening agent. This type of sweetening agent includes dextrose, polydextrose, sucrose, maltose, dextrin, dried invert sugar, mannose, xylose, ribose, fructose, levulose, galactose, corn syrup, sorbitol, mannitol, xylitol, maltitol, isomaltitol, aspartame, neotame, saccharin and salts thereof, sucralose, and a mixture thereof. One or more sweetening agents optionally exist at a content of about 0.005-10%, and optionally about 0.01-1%.

Optional Active Substance

The composition of the present disclosure optionally includes one or more additional active substances, capable of being used for preventing or treating oral sclerous or soft tissue diseases or disorder, preventing or treating physiological disorder or diseases, or providing makeup benefits. In various implementations, an active agent is an “oral care active agent” capable of being used for treating or preventing disorder or providing makeup benefits in the oral cavity (for example, for teeth, gums or other oral sclerous or soft tissues). The oral care active agent capable of being used in the present disclosure includes a coloring agent, a corrosion inhibitor, an anticaries agent, an antiplaque agent, an anticalculus agent, an anti-inflammatory agent, a deodorant, a desensitizer, or combination thereof, etc.

The active agent capable of being used in the present disclosure optionally exists in the composition of the present disclosure at a safe and effective amount. The “safe and effective” amount of the active agent is a sufficient amount to generate a required treatment or prevention effect in human or lower animal subjects using the active agent without inappropriate adverse effects (such as toxicity, irritation or allergic reaction). During application in a mode of the present disclosure, a reasonable benefit/risk ratio is realized. The specific safe and effective amount of the active agent will change along with factors of specific diseases to be treated, the body health condition of the subject, properties of parallel treatment (if any), the specific used active agent, a specific dose form, a used carrier and a required dosage regimen.

pH Regulator

pH regulators capable of being used in the present disclosure include a pH lowering acidifier, a pH raising basifier, and a buffering agent for controlling the pH in a required range, and for example, include one or more compounds selected from an acidifier, a basifier and a buffering agent so as to provide the pH of approximately 2-10. Any oral acceptable pH regulator can be used, including carboxylic acid, phosphoric acid and sulfonic acid, acidic salts (such as monosodium citrate, disodium citrate and monosodium malate), alkali metal hydroxides such as sodium hydroxide, carbonates such as sodium carbonate, bicarbonate, sesquicarbonate, borate, silicate and phosphate (such as monosodium phosphate, trisodium phosphate and polyphosphates). One or more pH regulators optionally exist at a total amount of effectively maintaining the composition at the oral acceptable pH range.

Water

In various implementations of the present disclosure, water also exists in the oral composition, and in at least one embodiment, deionized water without organic impurities is used. The water added is free water except for water introduced together with other substances, such as sorbitol. The water generally accounts for 10-50%, preferably 15-35% of the toothpaste composition of the present disclosure.

Surfactant

In further embodiments, the oral care composition of the present disclosure is a surfactant-free composition. A term “surfactant” of the present disclosure is also called as an interfacial agent, and is a substance capable of lowering liquid surface tension or interface tension between two phases. The surfactant has amphipathicity, and includes hydrophilic groups and lipophilic groups. Examples of the hydrophilic groups include, but are not limited to an amino group, carboxyl, a sulfonic group, hydroxyl, etc. Examples of hydrophobic groups include, but are not limited to C8 and above aliphatic alkanes, alkenes, alkynes, aromatic hydrocarbons, etc. Examples of common use surfactants include, but are not limited to natural surfactants or their derivatives, and synthetic surfactants. Examples of the natural surfactants or their derivatives include surfactants based on protein.

Exemplary anion surfactants, for example, water-soluble salts of higher fatty acid monoglyceride monosulfates, such as sodium salts of monosulfated monoglyceride of hydrogenated coconut oil fatty acids, such as sodium N-methyl N-cocoyl taurate, and sodium cocoglyceride sulfate; higher alkyl sulfates, such as sodium lauryl sulfate; higher alkyl ether sulfates, such as sodium laureth-2 sulfate; higher alkyl aryl sulfonates, such as sodium dodecylbenzene sulfonate (sodium lauryl benzene sulfonate); and higher alkyl sulfoacetates, such as sodium lauryl sulfoacetate (sodium dodecyl sulfoacetate), higher fatty acid esters of 1,2-dihydroxypropanesulfonic acid, sulfolaurate and sodium lauryl sarcosinate.

According to the cation surfactant of the present disclosure, the cation surfactant can be broadly defined as a derivative of an aliphatic quaternary ammonium compound having a C8-C18 long alkyl chain, such as lauryl trimethyl ammonium chloride, cetyl pyridinium chloride, cetyl trimethyl ammonium bromide, diisobutylphenoxyethyl dimethyl benzyl ammonium chloride, coco alkyl trimethyl ammonium nitrite, cetyl pyridinium fluoride and a mixture thereof.

Exemplary nonionic surfactants can be broadly defined as a compound produced by condensing an alkylene oxide group with an organic hydrophobic compound capable of being an aliphatic or alkyl aromatic compound in nature. Examples of appropriate nonionic surfactants include, but are not limited to a polyethylene oxide condensate of alkyl phenol, a condensation product derived from the a reaction product of ethylene oxide, propylene oxide and ethylenediamine, an ethylene oxide condensate of aliphatic alcohol, long-chain tertiary amine oxide, long-chain tertiary phosphine oxide, long-chain dialkyl sulfoxide and a mixture of these type of substances, such as poloxamer, polysorbate and polyethylene glycol hydrogenated castor oil.

Exemplary amphoteric surfactants include betaine (such as cocamidopropyl betaine); derivatives of aliphatic secondary and tertiary amines, wherein aliphatic groups can be linear or branched, one of aliphatic substituents includes about 8-18 carbon atoms, and one includes an anion water-solubilizing group (such as carboxylate, sulfonate, sulfate, phosphate, or phosphonate); and a mixture of this type of materials.

In further embodiments, the present application includes no preservative and no bacteriostatic agent. The preservative and the bacteriostatic agent are selected from alcohols, formaldehyde donors and aldehyde derivatives, benzoic acid and derivatives thereof, and other organic compounds, such as phenoxyethanol and benzyl alcohol, imidazolidinyl urea, parabens, bronopol, carbazone, and quaternary ammonium salts.

Not constrained by a theory, the microbial growth is inhibited by controlling the water activity of the oral care composition through the high-content humectant, such as water-soluble small molecules such as glycerol, sorbitol, xylitol and isomaltitol, and natural high-molecular polymers. The water activity of the oral care composition of the present disclosure (as the toothpaste) is less than 0.75, preferably less than 0.65, and particularly less than 0.6.

### BRIEF DESCRIPTION OF THE DRAWINGS

FIG. 1 shows foaming capability comparison of toothpaste.

FIG. 2 shows the mung beans condition after 15 hours of M1 experiment.

FIG. 3 shows the mung beans condition after 15 hours of M2 experiment.

FIG. 4 shows the mung beans condition after 15 hours of M3 experiment.

FIG. 5 shows the mung beans condition after 15 hours of M4 experiment.

FIG. 6 shows the mung beans condition after 15 hours of M5 experiment.

FIG. 7 shows the mung beans condition after 15 hours of M6 experiment.

FIG. 8 shows the mung beans condition after 15 hours of Embodiment 1 #experiment.

FIG. 9 shows the mung beans condition after 15 hours of Embodiment 1 #experiment.

FIG. 10 shows the mung beans condition after 15 hours of Embodiment 1 #experiment.

FIG. 11 shows the mung beans condition on the fifth day of Embodiment 1 #experiment.

FIG. 12 shows the mung beans condition on the seventh day of Embodiment 1 #experiment.

### DESCRIPTION OF THE EMBODIMENTS

The present disclosure will be more clearly understood by reading the following embodiments. These embodiments are not intended to limit the present disclosure but are merely exemplary implementations of the present disclosure. If not specifically specified, the component content in the following embodiments is mass percent.

An oral care composition was prepared according to the following proportioning ratio:

Formula of Embodiment 1 #: 55 of sorbitol (70% sorbitol aqueous solution), 22 of dicalcium phosphate, 5.5 of glycerol, 5.5 of xylitol, 2.2 of cellulose, 2.2 of a starch, 4.6 of a lactobacillus fermentation compound, and 3.0 of MC(X1).

Formula of Embodiment 2 #: 52.5 of sorbitol (70% sorbitol aqueous solution), 22 of dicalcium phosphate, 8.0 of glycerol, 5.5 of xylitol, 2.2 of cellulose, 2.2 of a starch, 4.6 of a lactobacillus fermentation compound, and 3.0 of MC (X2).

Formula of Embodiment 3 #: 50 of sorbitol (70% sorbitol aqueous solution), 22 of dicalcium phosphate, 10.5 of glycerol, 5.5 of xylitol, 2.2 of cellulose, 2.2 of a starch, 4.6 of a lactobacillus fermentation compound, and 3.0 of MC (X3).

Oral care compositions were prepared according to the following proportioning ratio:

50 of sorbitol (70% sorbitol aqueous solution), 22 of dicalcium phosphate, 10.5 of glycerol, 5.5 of xylitol, 2.2 of cellulose, 2.2 of a starch, 4.6 of a lactobacillus fermentation compound, and 3.0 of MC (Xn*);
note: Xn represents MC of different models, and n is 4, 5, 6, etc.

Surface Tension Test Method:

MC was prepared into 0.1% of an aqueous solution, and the surface tension of the solution was determined through a JYW-200C fully automatic interface tensiometer.

Foam Test Method:

Foaming performance of toothpaste was determined in the following mode: a section of toothpaste being 4 cm in length was squeezed onto a rough stone surface. The rough stone was wetted in advance, and a toothbrush was wetted for repeated brushing for 1 min by simulating a real toothbrushing frequency. The foaming performance of the toothpaste was observed, and was evaluated by a number of 0-4. When the composition was used as the toothpaste, foaming performance of 2 represented a certain degree of foaming, 3 and 4 represented good foaming effect and 0 represented foaming incapability.

TABLE 1
Surface tension of MC and foaming capability of compositions

| MC | X1 | X2 | X3 | X4 | X5 |
|---|---|---|---|---|---|
| Surface tension (mN/m) | 52.66 | 55.52 | 58.46 | 47.90 | 53.78 |
| Foaming capability | 4 | 4 | 3 | 4 | 4 |
| MC | X6 | X7 | X8 | X9 | X10 |
| Surface tension (mN/m) | 48.04 | 50.71 | 72.79 | 53.57 | 71.90 |
| Foaming capability | 4 | 4 | 0 | 4 | 0 |

Comparative example 4 #: a commercially available toothpaste containing a surfactant, the surfactant is sodium lauroyl sarcosinate.

Results were as shown in FIG. 1 and Table 1. When the surface tension of the aqueous solution of 0.1% selected MC was 47-59 mN/m, the toothpaste of the present disclosure had almost identical foaming effect to surfactant-containing toothpaste sold in the market.

Stability Experiment:

TABLE 2
Water activity Aw of three toothpaste compositions

| Toothpaste | | | |
|---|---|---|---|
| composition | Embodiment 1# | Embodiment 2# | Embodiment 3# |
| Water | 0.68 | 0.64 | 0.60 |
| activity Aw | | | |

Bacteria culture medium of preservative effect challenge experiment: lecithin tween 80 nutrient agar (from Hopebio): 24.0 g of the culture medium (that is lecithin tween 80 nutrient agar) was added into a 1000 ml conical flask, was heated, boiled to a boiling state, and dissolved into 500 ml of distilled water. High-pressure sterilization was performed for 20 min at 121° C. for use.

Fungus culture medium: potato dextrose culture medium (PDA), and Bengal red agar culture medium (from Beijing Luqiao): 18.3 g of the culture medium (one of the above two culture medium, in this embodiment, the Bengal red agar culture mediumis used) was added into a 1000 ml conical flask, was dissolved in 500 ml of distilled water, was heated and boiled to a boiling state until complete dissolution, and high-pressure sterilization was performed for 15 min at 121° C. for use.
Normal saline: high-pressure sterilization was performed for 15 min at 121° C. for use.
Tested strains:
Escherichia Coli, ATCC 8739
Pseudomonas Aeruginosa, ATCC 9027
Staphy Lo Coccus Aureus, ATCC 6538
Candida Albicans, ATCC 10231
Aspergiblus Niger, ATCC 16404

Experiment Method:

This experiment referred to microbial antiseptic efficacy test method of United States Pharmacopeia USP39<51>. That is, 30 g of each sample to be tested was weighed and added into a sterilized sample bottle. A quantitative mixed bacterial suspension (the above five strains) was added. An initial bacterium (Escherichia Coli, Pseudomonas Aeruginosa and Staphy Lo Coccus Aureus) adding concentration of bacteria in the sample was 10⁶ cfu/g. An initial bacterium adding concentration of Candida Albicans and Aspergiblus Niger was 10⁵ cfu/g. Then, sufficient and uniform mixing was performed. An opening of the sample bottle was sealed by opening sealing glue, and the sample bottle was put in an incubator about 28° C. Sampling was respectively performed at 7th, 14th, 21st and 28th days of inoculation for analysis.

Evaluation Standard:

A total bacteria number was from an initial value to a bacteria number logarithmic reduction value of not be less than 1.0 on the 7th day; the total bacteria number was from an initial value to a bacteria number logarithmic reduction value of not be less than 3.0 on the 14th day; and additionally, the bacteria number was not increased from the 14th day to the 28th day. A total number of Candida Albicans and Aspergiblus Niger from the initial to the 7th day, the 14th day and the 28th day was not increased.

TABLE 3
Colonies number change with time

| | | Colonies number/(cfu · g⁻¹) | | | | | |
|---|---|---|---|---|---|---|---|
| Strain | Number | Day 0 | Day 1 | Day 7 | Day 14 | Day 21 | Day 28 |
| Mixed | Embodiment 1# | 4.1 × 10⁶ | 3.7 × 10⁴ | <10 | <10 | | |
| bacteria | Embodiment 2# | 3.9 × 10⁶ | 4.2 × 10⁴ | <10 | <10 | | |
| | Embodiment 3# | 2.9 × 10⁶ | 1.8 × 10⁶ | 1.0 × 10⁴ | <10 | | |
| Mixed | Embodiment 1# | 3.6 × 10⁵ | 3.3 × 10⁴ | <100 | <10 | | |
| fungi | Embodiment 2# | 3.7 × 10⁵ | 4.3 × 10⁴ | <100 | <10 | | |
| | Embodiment 3# | 1.9 × 10⁵ | 1.8 × 10⁴ | 4.7 × 10² | <100 | | |

Samples of preservative-free systems of toothpaste compositions of the three embodiments were subjected to microbiological challenge experiments. Results showed that the compositions could pass the challenge experiments. Therefore, it could be seen that according to the present disclosure, a preservative effect could be achieved by controlling the water activity Aw to be less than 0.7.

Safety Experiment

Mung Bean Sprouting Experiments

The life of a plant begins with the sprouting of seeds. Water is a necessary condition for the germination and the growth of bean sprouts. The presence of pollutants (chemical components in toothpaste) in water can inhibit seeds sprouting and bud (seedling) growth. This experiment analyzes the safety or potential harm of toothpaste ingredients through the effect of toothpaste-water solution on the sprouting of mung bean seeds.

(1) Tested Toothpaste Samples

TABLE 4
Tested toothpaste samples

| Number | Surfactants in toothpaste |
|---|---|
| M1 | Sodium lauryl sulfate |
| M2 | Sodium lauryl sulfate, Cocoamidopropyl betaine |
| M3 | Lauryl glucoside, Sodium cocoyl glutamate |
| M4 | Sodium lauryl sulfate, Sodium lauroyl sarcosinate |
| M5 | Cocoamidopropyl betaine, Sodium methyl |
| | cocoyl taurate |
| M6 | Steareth-30 |
| Embodiment 1# | — |
| Embodiment 2# | — |
| Embodiment 3# | — |

Note:
M1-M6 were toothpaste on sale in the market, “—” represented surfactant-free.

(2) Test Method:

The toothpaste was diluted to 20 times with pure water in a culture dish, 6 healthy mung beans were put into the culture dish, the germination and the growth of bean sprouts were observed, to test the safety or potential toxicity of the toothpaste ingredients.

(3) Test Results:

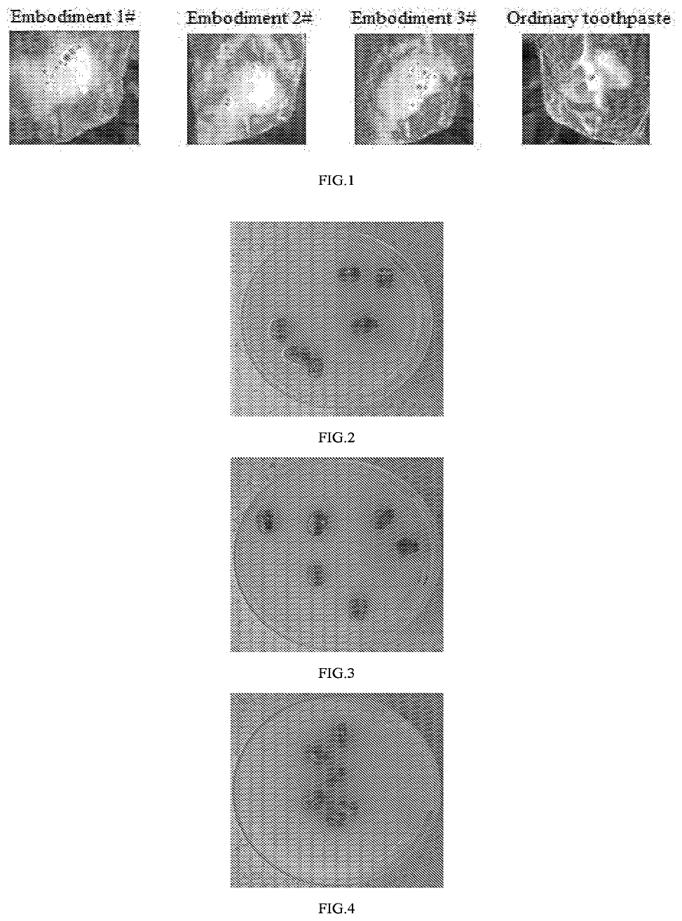

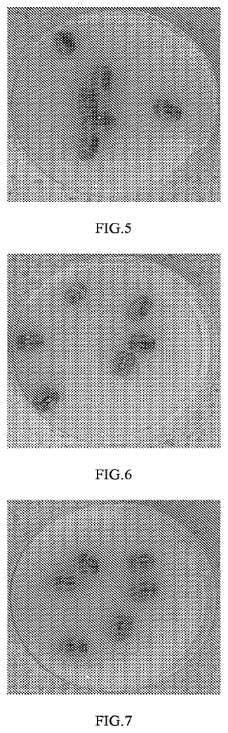

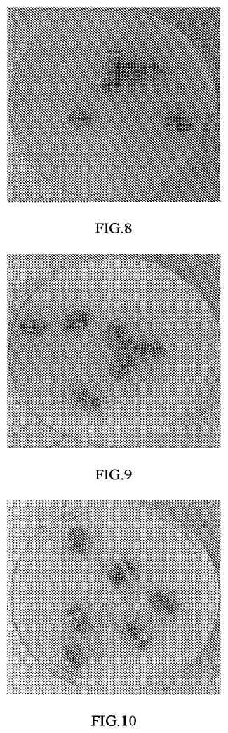

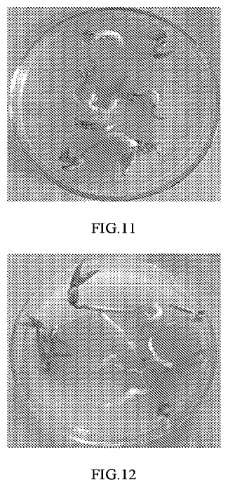

TABLE 5
Test results

| Number | Surfactants in toothpaste | Test results |
|---|---|---|
| M1 | Sodium lauryl sulfate | did not sprout (FIG. 2) |
| M2 | Sodium lauryl sulfate | did not sprout (FIG. 3) |
| | Cocoamidopropyl betaine | |
| M3 | Lauryl glucoside | did not sprout (FIG. 4) |
| | Sodium cocoyl glutamate | |
| M4 | Sodium lauryl sulfate | did not sprout (FIG. 5) |
| | Sodium lauroy1 sarcosinate | |
| M5 | Cocoamidopropyl betaine | did not sprout (FIG. 6) |
| | Sodium methyl cocoyl taurate | |
| M6 | Steareth-30 | did not sprout (FIG. 7) |
| Embodiment | — | All the beans sprouted |
| 1# | | within 15 hours (FIG. 8) |
| Embodiment | — | All the bean sprouted |
| 2# | | within 15 hours (FIG. 9) |
| Embodiment | — | All the beans sprouted |
| 3# | | within 15 hours (FIG. 10) |

Within the scope of the study, none of the mung beans put in toothpastes sold in the market sprouted (FIG. 2-7); all the mung beans put in the surfactant-free toothpaste provided by present disclosure sprouted 100% (FIGS. 8-10), and the subsequent bean sprouts grow well (FIG. 11. FIG. 12).

1. A foamable oral care composition, comprising a methyl cellulose, a surface tension of the methyl cellulose in an aqueous solution at a concentration of 0.1 mass % is less than 60 mN/m, preferably 47-59 mN/m; and a content of the methyl cellulose in the composition is 0.1-10 weight %, preferably 0.5-10 weight %, more preferably 1-7 weight %, especially preferably 2-5 weight %, and particularly 2.5-4.0 weight %, based on the weight of the composition.

2. The oral care composition according to claim 1, comprising:
(a) 10-50% (w/w/) of an abrasive, preferably 12-40% (w/w/), especially preferably 15-30% (w/w/), and particularly 18-25% (w/w/);
(b) 5-50% (w/w/) of a humectant, preferably 10-45% (w/w/), especially preferably 15-40% (w/w/), and particularly 18-30% (w/w/);
(c) 0.005-10.0% (w/w/) of a flavoring agent, preferably 0.1-5.0% (w/w/), and more preferably 0.5-1.0% (w/w/); and
(d) 0.01-10% (w/w/) of a functional additive, preferably 0.1-5% (w/w/), and more preferably 1.0-2.0% (w/w/).

3. The oral care composition according to claim 2, wherein, the abrasive is selected from silicon dioxide, such as silica gel, silicon dioxide hydrate or precipitated silicon dioxide; aluminum oxide; insoluble phosphate, calcium carbonate or combinations thereof.

4. The oral care composition according to claim 2, wherein, the humectant is selected from glycerol, sorbitol, xylitol, polyethylene glycol or combinations thereof.

5. The oral care composition according to claim 2, wherein, the flavoring agent is selected from a perfume and/or a sweetening agent.

6. The oral care composition according to claim 2, wherein, the functional additive is selected from a coloring agent, a corrosion inhibitor, an anticaries agent, an antiplaque agent, an anticalculus agent, an anti-inflammatory agent, a deodorant, a desensitizer or combinations thereof.

7. The oral care composition according to claim 1, comprising:
(a) 10-50% (w/w/) of an abrasive, preferably 12-40% (w/w/), especially preferably 15-30% (w/w/), and particularly 18-25% (w/w/);
(b) 5-50% (w/w/) of a humectant, preferably 10-45% (w/w/), especially preferably 15-40% (w/w/), and particularly 18-30% (w/w/);
(c) 2-20% (w/w/) of cellulose, preferably 4-16% (w/w/), especially preferably 6-12% (w/w/), and particularly 8-10% (w/w/);
(d) 5-30% (w/w/) of a lactobacillus fermentation compound, preferably 8-26% (w/w/), especially preferably 12-22% (w/w/), and particularly 14-18% (w/w/);
(e) 1-12% (w/w/) of a starch, preferably 2-10% (w/w/), especially preferably 3-8% (w/w/), and particularly 4-6.0% (w/w/).

8. The oral care composition according to claim 1, comprising no surfactant, the surfactant comprises an anion surfactant, a cation surfactant, an amphoteric surfactant and a nonionic surfactant.

9. The oral care composition according to claim 1, comprising no preservative.

10. The oral care composition according to claim 1, wherein, the oral care composition has a water activity of less than 0.75, preferably less than 0.65, particularly less than 0.6.

11. The oral care composition according to claim 3, wherein, the humectant is selected from glycerol, sorbitol, xylitol, polyethylene glycol or combinations thereof.

12. The oral care composition according to claim 3, wherein, the flavoring agent is selected from a perfume and/or a sweetening agent.

13. The oral care composition according to claim 4, wherein, the flavoring agent is selected from a perfume and/or a sweetening agent.

14. The oral care composition according to claim 3, wherein, the functional additive is selected from a coloring agent, a corrosion inhibitor, an anticaries agent, an antiplaque agent, an anticalculus agent, an anti-inflammatory agent, a deodorant, a desensitizer or combinations thereof.

15. The oral care composition according to claim 4, wherein, the functional additive is selected from a coloring agent, a corrosion inhibitor, an anticaries agent, an antiplaque agent, an anticalculus agent, an anti-inflammatory agent, a deodorant, a desensitizer or combinations thereof.

16. The oral care composition according to claim 5, wherein, the functional additive is selected from a coloring agent, a corrosion inhibitor, an anticaries agent, an antiplaque agent, an anticalculus agent, an anti-inflammatory agent, a deodorant, a desensitizer or combinations thereof.

17. The oral care composition according to claim 2, comprising no surfactant, the surfactant comprises an anion surfactant, a cation surfactant, an amphoteric surfactant and a nonionic surfactant.

18. The oral care composition according to claim 2, comprising no preservative.

19. The oral care composition according to claim 10, wherein, the oral care composition has a water activity of less than 0.65.

20. The oral care composition according to claim 10, wherein, the oral care composition has a water activity of less than 0.6.
